(19) United States
(12) Patent Application Publication
(10) Pub. No.: US 2004/0131754 A1
(43) Pub. Date: Jul. 8, 2004

(54) Antibiotic calcium phosphate coating

(75) Inventors: Joseph Zitelli, River Edge, NJ (US); Paul Higham, Ringwood, NJ (US); Paresh Dalal, Greenville, NC (US); Monica Hawkins, Denville, NJ (US); Renwen Zhang, Wayne, NJ (US)

Correspondence Address: LERNER, DAVID, LITTENBERG,, KRUMHOLZ & MENTLIK, 600 SOUTH AVENUE WEST, WESTFIELD, NJ, 07090, US

(73) Assignee: Howmedica Osteonics Corp., Allendale, NJ

(21) Appl. No.: 10/611,147

(22) Filed: Jul. 1, 2003

Related U.S. Application Data
(63) Continuation-in-part of application No. 10/001,525, filed on Oct. 24, 2001, now Pat. No. 6,596,338.
(60) Provisional application No. 60/396,405, filed on Jul. 15, 2002.

(51) Int. Cl. A61L 2/00

(52) U.S. Cl. 427/2.24

(57) ABSTRACT

A process for applying a coating having a therapeutic agent such as an antibiotic or a bone morphogenic protein such as OP-1 protein to an implant uses the high surface area of a calcium phosphate coated metal implant as a repository for the therapeutic agent. The implant is coated with one or more layers of calcium phosphate minerals such as hydroxyapatite. After the crystalline layer is applied, which is usually done within an aqueous solution, the implant is dried and packaged. Immediately prior to implantation, the implant is removed from the package and the crystalline layer of calcium phosphate is wetted with an aqueous solution containing the therapeutic agent.

### CROSS-REFERENCE TO RELATED APPLICATIONS

This application is a continuation-in-part of U.S. Ser. No. 10/001,525 filed Oct. 24, 2001. This application claims priority of Provisional Application No. 60/396,405 filed Jul. 15, 2002 which is incorporated herein by reference for the additional matter added.

### BACKGROUND OF THE INVENTION

The field of this invention relates to mineralized coatings of prosthetic devices. More particularly the invention relates to a porous calcium phosphate mineral coated prosthesis which includes a coating having a therapeutic agent, such as an antibiotic in water, absorbed therein.

The use of prosthetic devices for treatment of bone injuries/illnesses is continuously expanding with an increasingly active and aging population. The use of bone replacements for bone fractures, removal of bone, or the use of supports for weakened bone requires that the artificial bone replacement form a strong joint or bone with natural bone to insure the integrity of the structure. Bone is able to grow into adjacent structure, particularly where the adjacent structures are porous and compatible with the bone. However, not only must the bone be able to grow into a porous structure, but there must be bonding in a form which allows for a strong bond between the natural ingrown bone and the prosthetic device.

The key requirement for bony fixation of prosthetic implants is that bone grows onto and/or into the implant's surface. A number of studies have shown that calcium phosphate coatings, such as biological apatite, on Cobalt Chrome (Co—Cr) and Titanium (Ti)-alloy implants foster more rapid bony apposition than the bare surfaced alloys alone.

Biological apatite Ca₁₀(PO₄)₆(OH)₂ is one of the major compounds occurring in human bones and teeth. A synthetic form of this mineral, hydroxyapatite (HA) is very similar to the natural occurring apatite. This similarity between synthetic HA and naturally occurring apatite has led scientists to pursue the use of HA with dental and orthopedic implants. Coating with HA or other crystalline calcium phosphate produces an implant that readily integrates with surrounding bone and tissue after being implanted.

Some of the first dental and orthopedic implants attempting to employ synthetic apatite were completely formed from sintered or plasma sprayed HA.

Plasma spraying is one process known for coating metallic implants with HA. During this process, a stream of mixed gases passes through a high temperature electric arc that ionizes the gases into a plasma flame. Thereafter, crystalline HA feedstock powder is fed into the stream and then impinged in a molten state onto the outer surface of the implant. The spray adheres to the surface and forms a relatively thin coating of ceramic HA.

HA coated implants exhibit the advantages of both purely metallic implants and purely HA implants. As such, these implants are strong, and bone tissue tends to form a strong bone interface with the surface of the coating and thus promote biocompatibility and osseointegration. Unfortunately, plasma spraying results in several important disadvantages.

Plasma spraying exposes HA to extremely high temperatures that, in turn, induce unwanted changes in morphology and chemical composition. These changes pose particular problems. In particular, it is known that highly crystalline HA has an in vitro stability that is much higher than non-crystalline HA. HA feedstock of a good quality does have a completely crystalline form before it is sprayed. The temperatures associated with plasma spraying, though, cause the HA to partially transform from its pure and crystalline form to one having a much less crystalline structure. This non-crystalline form of HA is commonly referred to as amorphous calcium phosphate (ACP). During plasma spraying, crystalline HA feedstock is also partially converted into other crystalline compounds, such a tri-calcium phosphate (TCP including α-TCP and β-TCP), tetracalcium phosphate (TTCP), and calcium oxide (CaO). Collectively, these impurities may be referred to as crystalline soluble phases because their solubility in aqueous solutions is substantially higher than that of crystalline HA. Thus, a process for low temperature deposition of crystalline HA was desired.

In addition, the success of a cementless arthroplasty prosthesis depends on the ingrowth of bone from the surrounding osseous environment and it is known that providing a hydroxyapatite (HA) coating on the implant surface can enhance this integration. With the development of recombinant human protein techniques, there is an increasing interest in combining HA with growth factors to transform the osteoconductive HA coating into an osteoinductive coating. Osteogenic protein-1 (OP-1®), a registered trademark of Stryker Corporation, is a 36 kD homodimer protein in the TGI-β super-family which includes a group of up to fifteen (15) proteins referred to as BMPs (bone morphogenic proteins). OP-1® Osteogenic graft material is also a known bone morphogenic protein and has ectopic bone induction pr formation capacity. Synthetic OP-1® Osteogenic graft material is set forth in U.S. Pat. No. 4,968,590, the teachings of which are incorporated herein by reference. Application of this protein on implant surface has been found to induce bone formation on the surface of the implant. However, it has been found to induce to augment the integration process necessitates a degree of retentive capability from the coating material so that the protein can elicit the required response for an adequate duration of time. It has been reported that particles of hydroxyapatite, functioning as BMP carriers, have induced ectopic bone formation. OP-1® Osteogenic graft material is currently used in orthopedics as an implant device consisting of OP-1® Osteogenic graft material mixed with purified bovine bone-derived Type I collagen. The efficacy of OP-1® Osteogenic graft material has been shown in cases where it has been applied onto a hydroxyapatite-coated implant that was used in the presence of a bone defect. In the cases of HA-coated press-fit implants, it is desirable to have OP-1® Osteogenic graft material loaded onto hydroxyapatite.

Peri-apatite™ (PA) is a unique coating technology, which forms a dense crystalline network of HA on the implant surface with a surface area significantly greater than traditional flame plasma sprayed HA coatings. Peri-apatite™ is a trademark of Stryker Corporation. The high surface area of this dense crystalline network provides the binding capacity for the OP-1® Osteogenic graft material solution, when introduced by, for example, simply soaking onto the surface.

Crystalline calcium phosphate coatings are preferably produced in a low temperature one or multi-step process which provides for a strong adherent uniform thin coating of crystalline hydroxyapatite on a substrate surface, where the coating has long needles or whiskers, which appear to induce bone ingrowth and strong bonding between natural bone and the coating via bone ingrowth and opposition on a pore comprising implant.

The coatings are found to have a high hydroxyapatite Ca₁₀(PO₄)₆(OH)₂ surface area because of the fibrous hydroxyapatite crystals. The surface area will generally range from about 1-25 m²/cm² of area. The coatings may be as thin as about 2 μm, preferably being at least about 5 μm (μm=microns), and more preferably at least about 10 μm, and may range to 40 μm thick or greater, depending upon need. Usually, a relatively thin coating will be employed to avoid thick brittle ceramic interfaces between the substrate and the ductile bone. The process taught in U.S. Pat. Nos. 5,164,187 and 5,188,670, the teachings of which are incorporated herein by reference, may produce such coatings.

The single crystals or whiskers, which are produced by the method of U.S. Pat. No. 5,164,187, will generally range from about 0.01 microns to 20 microns in diameter and about 1 micron to 40 microns in length. The composition will usually be substantially homogenous (≧95%), mineralogically pure i.e., highly crystalline (same crystal structure) (≧90%) and substantially homogenous morphologically, generally varying by no more than ±20% from the average of each dimension.

The crystalline hydroxyapatite has a net positive charge at physiologic pH which attracts charged proteins, such as collagen or other exogenous or endogenous proteins, which may serve as growth factors, chemoattractants, and the like. Thus, the coating may provide for the presence of such products on the surface of the hydroxyapatite. The exceptionally high surface of this coating presents orders of magnitude more binding surface than the uncoated implant or the conventional calcium phosphate coatings. Specifically, it has been found that plasma sprayed HA coatings would not bind to a solution or suspension of antibiotic.

The calcium phosphate coatings (PA) may be applied to solid surfaces, porous surfaces, etched surfaces, or any other type of surface. Because the coating is applied in a liquid medium which is able to penetrate channels, pores, indentations and other structural features, a uniform coating can be obtained which can coat substantially the entire surface, without leaving exposed areas. The subject process finds particular application with devices involving fine bead layers, where the beads will be two or more layers, requiring that at least about two layers of the beads be penetrated and coated with the hydroxyapatite or its analog. Thus, penetrations are achieved in a porous substrate, such as is used in prosthesis devices, of at least about 0.5 mm, more usually at least about 1 mm.

### SUMMARY OF THE INVENTION

It is an object of the invention to provide a versatile and simple method of applying therapeutic agents to a calcium phosphate surface found by precipitation prior to implantation of a coated implant.

It is yet another object of the invention to provide a simple and fast method of providing a calcium phosphate coated surface with doses of water-soluble antibiotics prior to implantation of the implant.

These and other objects are achieved by a method where applying the therapeutic agent, especially an antibiotic, to the implant comprising coating the implant with preferably at least two layers of crystalline hydroxyapatite by precipitating the hydroxyapatite or calcium phosphate from solutions. The implant is then dried and packaged. In a preferred embodiment, immediately prior to implantation, the therapeutic agent or antibiotic is added to sterile deionized water or sterile water for injection and the implant is removed from the package and at least the hydroxyapatite or calcium phosphate surface thereof is immersed in the solution.

Alternatively, the antibiotic solution or suspension can be incorporated into the dried coating prior to packaging. The surgeon can then use the as supplied implant or add an additional coating of antibiotic to the HA antibiotic coated portion of the implant.

When applied in the operating room, the therapeutic solution may be pipetted into the calcium phosphate surface. The water and therapeutic agents may be added to the dried coating drop wise. The implant is then implanted in its wetted state. Alternately, the method for providing a therapeutic agent to an implant site includes providing the packaged implant coated with crystalline calcium phosphate or crystalline hydroxyapatite and the therapeutic agent by the same process as described above prior to packaging. If done in the operating room immediately prior to implantation, the calcium phosphate or hydroxyapatite coated implant is removed from the package and coated with an aqueous solution containing the therapeutic agent such as an antibiotic or a bone morphogenic protein. This can be done by immersing the calcium phosphate or hydroxyapatite coated implant into an aqueous solution of a therapeutic agent such as, for example, an antibiotic or bone growth stimulator such as bone morphogenic protein. Alternately, the aqueous solution may be pipetted or even poured over the surface. The implant may be either implanted in the bone canal in its wet condition or allowed to dry in air prior to implantation. The therapeutic agents, such as an antibiotic, may be either dissolved in the water to form the aqueous solution or may be suspended in the water to form the aqueous mixture that is placed on the calcium phosphate hydroxyapatite coating.

Useful antibiotics for use in this method are cefamandole, tobramycin, vancomycin, penicillin, cephalosporin C, cephalexin, cefaclor, cefamandole, ciprofloxacin and bisphosphonates.

### BRIEF DESCRIPTION OF THE DRAWINGS

FIG. 1 is a binding curve of OP-1® Osteogenic graft material on Peri-Apatite™ coated titanium disks;

FIG. 2 is a bone and marrow formation on a PA coated titanium disk treated with OP-1® Osteogenic graft material;

FIG. 3 is a time course graph of OP-1® Osteogenic graft material binding to PA-coated Ti disks;

FIG. 4 is a graph binding of OP-1® Osteogenic graft material to PA-coated Ti disks with different concentrations of OP-1® Osteogenic graft material;

FIG. 5 is a graph of the release of OP-1® Osteogenic graft material from PA-coated Ti disks; and

FIG. 6 is a cumulative release of OP-1® Osteogenic graft material from PA-coated Ti disks.

### DETAILED DESCRIPTION OF THE PREFERRED EMBODIMENTS

While a single layer of calcium phosphate may be applied the preferred method involves applying at least two layers of calcium phosphate to a metallic prosthesis. The first layer is of very small crystals achieved by providing conditions which result in a high density of heterogeneous nucleation sites, so that there is a large number of hydroxyapatite nucleation sites on the substrate. This is preferably followed by at least one additional coating under conditions that provide for a lower level of nucleation modulated crystal growth, so as to produce substantially larger crystals. Desirably, one or more additional coating layers are provided, where the conditions are the same or at even lower levels of nucleation than the second coating to produce larger size crystals as compared to the second coating. Usually, there will be not more than five coatings, preferably not more than about three coatings.

The first layer will generally be of a thickness in a range of about 0.01 microns to 10 microns. The second coating will generally be of a thickness in a range of about 1 micron to 40 microns, with crystals of a size in the length range of about 0.01 microns to 20 microns. The third and successive coatings will generally range as to each layer of a thickness in a range of about 1 micron to 40 microns, preferably about 5 microns to 10 microns, having crystals of a diameter of about 0.1 to 2 microns, and a length of about 1 to 10 microns, preferably about 0.1 to 1 micron in diameter, and a length of about 2 microns to 7 microns. The total thickness of the second and succeeding layers will generally be in the range of about 5 microns to 20 microns.

The various layers can be achieved, by varying the concentration of the reactants, the pH, temperature, manner of combining the reactants in the reactor, nature of the liquid flow, and the like. Preferably, the reactants and substrate will move in relation to one another, so that the substrate is continuously encountering a specific reaction mixture. Conveniently, the reaction mixture may be streamed past the substrate, using laminar or turbulent flow, preferably turbulent flow, either by providing for a tubular reactor with a reaction mixture which may be recycled and spent ingredients replenished, or by using a mixer, where the portion of the substrate to be coated is positioned at a site displaced from the center of the reactor and the reaction mixture continuously agitated with a stream flowing around the walls or the like. The specific conditions for the reaction mixture are determined by the flow conditions determined by reactant concentration, geometry of combination, fluid flow regime, vessel geometry, and the like. If desired, these conditions can be varied in a manner that allows the coating to be applied in one step.

To obtain the coating, vitallium (Co—Cr) or titanium implants are carefully cleaned and optionally passivated and introduced into a stainless steel processing tank comprising a 10% by weight ammonium acetate solution in deionized water (dh₂0). The implants are placed downstream from calcium and phosphate addition ports and rapidly agitated. The stainless steel tank is covered with a protective lid to reduce evaporative heat loss. When the ammonium acetate solution has been heated to 80° C., the pH monitored, so that the solution is maintained at 80° C. and ph 7.4. The coating process is begun when the temperature reached 80.0° C. and the solution was at pH 7.4. These conditions were maintained throughout the coating process.

Addition of the reactants, which preferably are 0.5M calcium acetate and 0.3M ammonium phosphate monobasic, is then begun at a rate to provide the desired calcium to phosphate ratio, while maintaining the pH by the addition of concentrated ammonium hydroxide. The addition is carried out over a period of two to four hours. The addition rate is varied to produce the desired thickness of coating.

The implant substrate may then be removed, washed and allowed to air dry. It may also be hot air dried. When the coating process is finished, the coatings are visually inspected for coating coverage and quality. If coating coverage or quality is unacceptable the coating may be removed and the process repeated. A description of this process may be found in the article “A Novel Method For Solution Deposition Of Hydroxyapatite On To Three Dimensionally Porous Metallic Surfaces: Peri-Apatite HA,” Mat. Res. Soc. Symp. Proc. Vol. 599, 2000 Material Research Society.

Once the crystalline coating has been applied to the prosthesis, the prosthesis is air dried, packaged and sterilized. The sterilized package implant is then ready for use in the operating room. Since the implant is not pre-coated with an antibiotic or other therapeutic agent such as OP-1® Osteogenic graft material or other Bone Morphogenic proteins (BMP), the surgeon can determine at the time of surgery whether an antibiotic or other agent is needed at all and, if so, what type of antibiotic or other agent would be best suited. The surgeon then uses the method of the present invention.

Because of the difference in surface area and surface energy between the non-crystalline plasma calcium phosphate coated implants and the implants in which the hydroxyapatite is precipitated from solution, the latter will readily absorb the water and antibiotic combination mixed by the surgeon at the time of surgery or mixed and applied prior to packaging. While any antibiotic or therapeutic agent may be used, whether water-soluble or in a suspension, the following examples illustrate a variety of antibiotics with various zones of inhibition (in millimeters) for infections indicated.

TABLE I

| | | Zone of Inhibition |
|---|---|---|
| Antibiotic | Salt | (mm) |
| Tobramycin | Sulfate | 10 |
| Vancomycin | Hydrochloride | 10.5 |
| Penicillin | Sodium Salt | 28 |
| Penicillin | Potassium Salt | 29 |
| Penicillin | Procaine | 26.5 |
| Penicillin | Benzathine | 22.5 |
| Cephalosporin C | Zinc Salt | 11.5 |
| Cephalexin | Hydrate | 19 |
| Cefaclor | Monohydrate | 20.5 |
| Cefamandole | Naftate | 25 |
| Ciprofloxacin | Hydrochloride | 15.5 |

Eleven antibiotics were used to overcome any concerns that there may be ionic interactions between the various salts (used to stabilize different antibiotics) and the calcium phosphate coating.

In all the tests, the results show that various antibiotics can be effectively incorporated into the precipitated crystalline coating and will be released rapidly at effective concentrations. There appears to be no interaction between the antibiotic salts and hydroxyapatite coatings that would bind the antibiotic to the coating, thereby preventing release.

The results are clinically significant because the proposed method will allow surgeons to prepare individual therapies for cultured bacteria at the time of revision or other surgery. The method does not preclude the use of thermally sensitive antibiotics, as would be the case when using exothermic bone cement as a carrier.

### EXAMPLE I

1.2 Grams of tobramycin sulfate was dissolved in 12 milliliters of sterile deionized water (100 mg/ml). Implants in the form of 12 mm diameter test coupons were of titanium that had been coated with crystalline hydroxyapatite as applied by the method of U.S. Pat. No. 5,164,187 were provided. Ten (10) μl of solution was pipetted onto the surface of each of the peripatetic (HA) coated disks and allowed to dry. Application of the liquid antibiotic solution or suspension can be performed via pipetting or using a syringe rather than immersion, which does not allow the same level of control of the therapeutic dosing. The test coupons were then placed into a buffered saline at 37° C. immediately after the absorption process was completed. It was determined that all the antibiotic was released into the buffered saline solution within twenty-four hours of immersion. Additional testing in the form of a modified Kirby Bauer susceptibility model was performed which showed that the released antibiotic remained biologically active and thus, was not affected by the absorption into the hydroxyapatite structure. In the well-known Kirby Bauer Susceptibility Model test organisms were propagated and handled in accordance with ATCC recommendations for broth media, agar, and incubation specifications. Staphylococcus aureus (ATCC strain #6538) was prepared by inoculating trypticase soy broth TSB and incubating at 37° C. for 24 hr. Microbial suspensions were adjusted to an absorbency of 0.325 using a spectrophotometer (wavelength=475 nm) and swabbed onto Mueller-Hinton agar plates. Each seeded plate was challenged with both an antibiotic containing HA disk and a non-antibiotic containing HA disk, which served as a control. Samples were tested in duplicate (i.e., two plates per organism). Following incubation for 24 hr. at 37° C., the plates were examined for zones of inhibition around the disks. The zone of inhibition is defined as the distance between the test disk and the edge of bacterial growth. It is common for a hazy, or “ghost” zone to exist between the areas of complete inhibition and full bacterial growth. This “ghost” zone is the result of partial inhibition and is not included in the measurement of the zone of inhibition. As a comparison, untreated test coupons coated with hydroxyapatite but not immersed in the antibiotic solution exhibited no antibacterial properties.

While the therapeutic solution was pipetted onto the surface the test coupon could have just as easily been applied by a syringe or immersed in the solution. If necessary, a series of applications of the therapeutic agents/drying can be done to increase the concentration of the agents.

### EXAMPLE II

Materials and Methods—Preparation of implant test articles: Titanium alloy (Ti6A14V) disks/coupons 5 mm in diameter and 3 mm thick were coated both sides with PA coating using the method of Example I. The thickness of the coating surface was determined by SEM to be nominally 20 μm to 50 μm.

Determination of the binding capacity of OP-1® Osteogenic graft material: A pre-mixed blend of unlabelled OP-1® Osteogenic graft material with iodine [¹²⁵ I] for tracking purposes was used. The OP-1® Osteogenic graft material in a lactose solution was pipetted on PA coated disks in triplicate. A range of OP-1® Osteogenic graft material incubation time and concentration treatment was applied to determine the time-course and binding capacity of the graft material on the PA coated disk. Following the incubation, each coupon was washed with lactose solution and the washes were collected in a scintillation vial. 10 ml of the scintillation cocktail was added to each test article and wash sample and the scintillation counts were measured in a liquid scintillation counter (LSC) to calculate the amount of OP-1® Osteogenic graft material bound to the PA coated disk.

Determination of the release rate of OP-1® Osteogenic graft material: A radiolabelled blend of OP-1® graft material at a pre-determined concentrations was pipetted on PA hydroxyapatite coated disks in triplicate. The disks were incubated at room temperature for 3 hours and then washed free of the unbound protein with lactose solution. They were then transferred into scintillation vials containing 5 ml phosphate buffered saline (PBS) and incubated with shaking for 7 days at 37° C. Periodic samples of the release medium were withdrawn and analyzed in a LSC to determine the amount of OP-1® Osteogenic graft material released in the PBS.

Six Ti disks were arranged in a humidified chamber, and 25 μl of labeled OP-1® Osteogenic graft material solution was added to the surface of each Ti disk. Activity controls were again prepared in the same manner as the previous phases. The disks were incubated at ambient temperature for 3 hours. Following 3 washes, three disks were placed in scintillation vials containing scintillant and incubated at ambient temperature overnight prior to counting. The remaining 3 disks were placed in scintillation vials containing 5 mL PBS. The disks were incubated and shaken continually for 1 hour at 37° C. The PBS was decanted into scintillation vials, scintillant was added, and the samples were counted. The disks were placed in fresh scintillation vials, and the same volume of PBS was added to the vials. At the next time point, the same process of decanting PBS and transferring disks was performed. This was done at each of the remaining time points, which occurred approximately every 24 hours for 7 days.

In-vivo study of OP-1® Osteogenic graft material coated implants: Freshly reconstituted OP-1® Osteogenic graft material in lactose solution was loaded onto each surface of the PA coated disk using sterile pipettes. The solution evenly spaced across the dry surface and was completely absorbed. Sixteen Long-Evans male rats with body weight 300-390 g were used in the study. The animals were anesthetized and an incision was made on the medial thigh to prepare intramuscular pouch within the adductor muscles for placement of the implant articles, on both contra-lateral limbs. A total of 32 implants were divided into 4 groups: (A) titanium disks; (B) titanium disks coated with PA, (C) titanium disks coated with PA plus OP-1® Osteogenic graft material (40 μg), (D) OP-1® Osteogenic graft material solution (40 μg). For Group C, the OP-1® Osteogenic graft material solution was directly pipeted on each Ti disk immediately before implantation. The solution was absorbed within 1 minute. For Group D, OP-1® Osteogenic graft material solution was dropped into the muscular pouch. The same amount of identical carrier solution for OP-1® Osteogenic graft material was loaded on disks of Groups A and B. After implantation, the pouch was closed with 3-0 chromic sutures, and the wound was closed in layers. The rats were housed individually and allowed unrestricted activity immediately after survey.

All the animals were sacrificed 4 weeks postoperatively. The complete limb was disarticulated from hip joint and x-rayed. The implants with their surrounding tissues were retrieved. All implants were processed for undecalcified thin section histology using EXAKT technique. The slides were stained with Stevenel's blue/van Gieson's picrofuchsin method for histological analysis. Bone was stained in red color and fibrous tissue in blue. Half of the specimens of Group D were decalcified and embedded in paraffin. Sections were stained with hematoxylin and eosin.

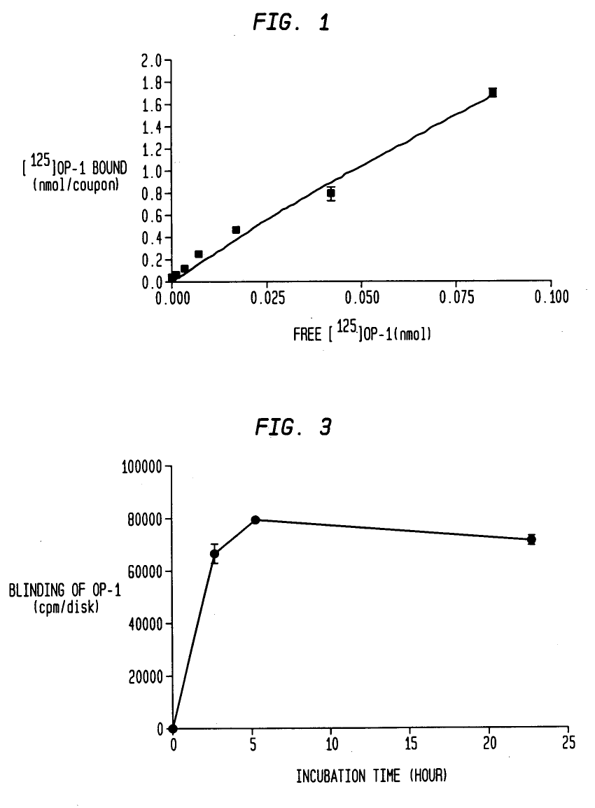

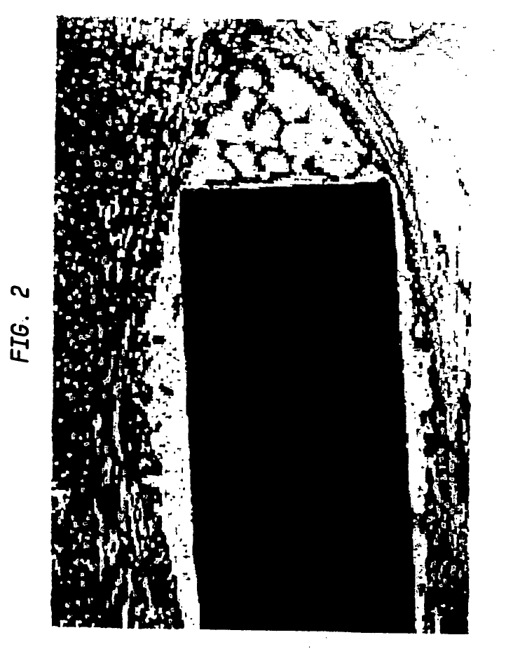

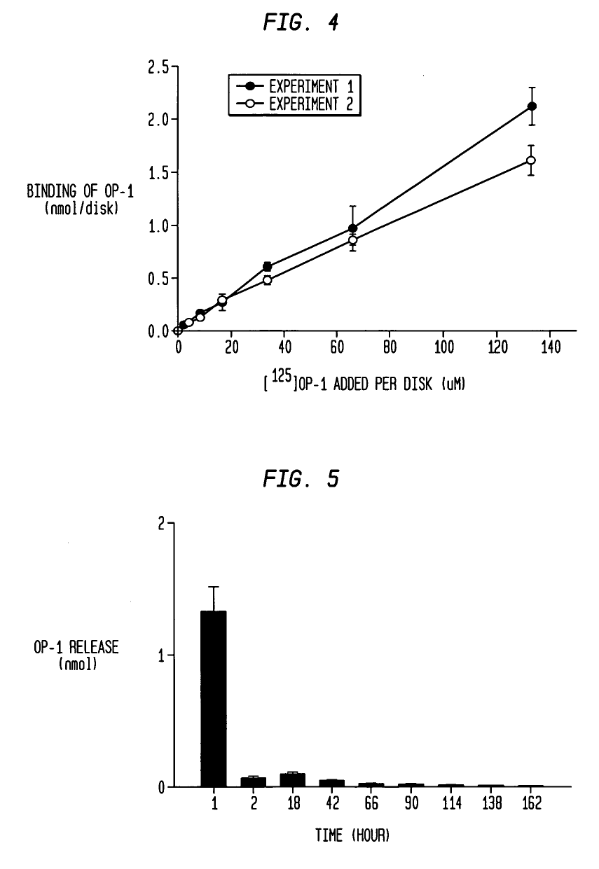

Results—As can be seen from FIG. 1, the binding of [¹²⁵I]OP-1® Osteogenic graft material to the PA coated coupons appeared to be linear up to 3 mg/ml[¹²⁵I]OP-1® Osteogenic graft material (79 μM). The binding of OP-1® Osteogenic graft material to the PA-coated Ti disk was increased with time, and the maximum binding was achieved at 5 hours (FIG. 3). The apparent reduction in binding after an overnight incubation was probably due to the variability in initial binding to the individual disk. The binding capacity of the PA-coated Ti disks were determined using the OP-1® Osteogenic graft material concentration from 70 μg/mL to 5 mg/mL. Data of two experiments showed that the binding of OP-1® Osteogenic graft material to the PA-coated disks appeared to be linear in the concentration range (FIG. 4). The binding did not reach a plateau, but 5 mg/mL (132 μM) was the highest concentration used. (Protein spontaneously precipitates if the concentration is over 5 mg/mL.)

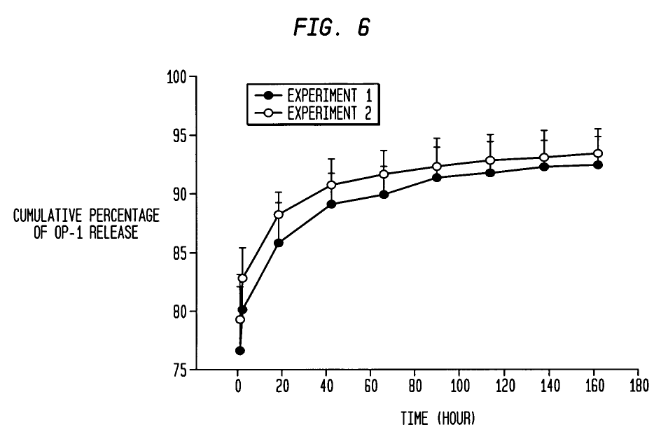

The percent release of OP-1® Osteogenic graft material from the PA coated coupons was very similar in the two experiments performed; 75-80% was released within the first hour of incubation at 37° C. (see FIG. 5). The mean bound of OP-1® Osteogenic graft material at time zero is 1.685 nmol/disk. The mean release of OP-1® Osteogenic graft material at 1 hour is 1.328 nmol/disk, about 79% of the value at time zero. Maximum cumulative release of about 92% was observed after 3 days incubation at 37° C. (see FIG. 6).

Radiographic evaluation revealed the bone formation inside the adductor muscles in all the specimens in Group C and Group D. The size of new bone in the muscles ranged from 5 to 14 mm in Group D. In Group C, the disks were surrounded by new bone, which also extended to some direction as far as 10 mm. None of the specimens in Group A and B showed any bone formation in the muscles. Histological examination confirmed the bone formation in the muscles, which had received OP-1® Osteogenic graft material. In Group D, all specimens exhibits bone nodule formation and was composed of trabeculae accompanied by marrow formation in all the specimens. Most traebecular bone showed the morphology of mature bone and there were residues of chondrocytes in the new bone. Marrow tissue was observed and expanded blood vessels were obvious. In all the specimens of Group C (PA coated Ti disks), bone formed a cylindrical ring surrounding the implant. Marrow tissue was present between the ring of bone and the implant at some places, while at other places the ossicles were directly in contact with the surface of the implant (FIG. 2). There was no inflammation in the specimens, which received OP-1® Osteogenic graft material. There was no inflammation in any of the specimens that received OP-1® Osteogenic graft material. Cellular transition from stromal cells of skeletal muscle to osteoblasts could be seen at the margin of new bone in a paraffin section. A column of hypertrophic chondrocytes was probably the residue of endochondral osteogenesis.

The disks in Group A and B were encapsulated by a thin layer of fibrous tissue. There was no bone or cartilage at the implanting site on any specimen not coated with OP-1® Osteogenic graft material.

Discussion—The incidence of bone formation was 100% in the two groups treated with OP-1® Osteogenic graft material and was zero in the two groups not treated with OP-1® Osteogenic graft material, indicating that OP-1® Osteogenic graft material can be loaded on Ti through the coating of PA while retaining its bioactivity. The advantage of PA coating over traditional plasma spray coating as a carrier is twofold. First, the plasma spray process is a line-of-sight process and can only coat the surface of an implant. PA is a solution precipitation technique that can deposit HA on the inside surface of a three-dimensional structure, such as porous coating that has been widely used in orthopedic implants. Second, the high temperature used in plasma spraying causes partial melting of HA, reducing the surface area of HA. On the other hand, solution precipitation generates HA crystals with a large surface area. A preliminary test showed that the disk with PA coating can be loaded with twice as much buffer solution as a same sized disk with plasma-sprayed HA coating. The large surface area of PA may also work as an adherent surface for the attachment of cells that respond to OP-1® Osteogenic graft material. The large surface area of the PA coating (3-10 square meters per gram) and its open, porous structure allows large surfaces for protein interaction. The results indicate adequate binding of OP-1® Osteogenic graft material and sufficient protein release to demonstrate the potential benefit of delivering the OP-1® Osteogenic graft material on PA coated prostheses to generate a robust integration of implant with the surrounding osseous tissue.

Hydroxyapatite combined with OP-1® Osteogenic graft material has been applied to implants before. Lind et al. reported a moderate bone induction of an OP-1® Osteogenic graft material and bovine collagen. That device is good for orthopedic implantation where defects are present. Noshi et al. loaded partially purified bovine bone morphogenic protein in solution on HA ceramic and let it dry for 3 days at room temperature prior to implantation. The authors were unable to demonstrate any bone-forming capability of the BMP/HA composites. This method of loading BMP not only raises the concern of protein degradation and implant contamination during the drying period, but also prevents surgeons from applying BMP immediately before implantation. The approach of the present invention makes it possible to apply OP-1® Osteogenic graft material directly on implants during surgery.

The in vitro binding assay indicates that the maximal bind of OP-1® Osteogenic graft material to PA is achieved 5 hours after loading. It appears necessary to leave a certain amount of time for the absorption of protein to HA. Lind et al., for example, incubated implants with TGF-β1 solution for 2 hours. However, an ectopic bone-forming assay shows that OP-1® Osteogenic graft material efficiently induces bone around the disks without any time for incubation.

The in vitro OP-1® Osteogenic graft material releasing assay shows that 75-80% of binding OP-1® Osteogenic graft material is released within the first hour. This is similar to the report by Lind et al. on the releasing of TGF-β from HA, where it was reported that 90% of TGF-β was released within 4 hours. It has been demonstrated that the local retention of implanted BMP is related to the characteristics of BMP and the carrier. It should be noted that the Ti disks used in this study were not porous coated. Had they been coated with beads or foam titanium, the inside surface would be coated with PA, thus greatly increasing the loading capacity of OP-1® Osteogenic graft material. Also, the PA crystals are in the nanometer range, which might create micro space on the surface of the implant and aid in the retention of OP-1® Osteogenic graft material. The longer retention time of OP-1® Osteogenic graft material could be translated into more potent bioactivity. Uludag et al. have reported that rhBMP's with a higher retention elicit more bone formation.

A quantitative analysis was not conducted for several reasons. The difference between the OP-1® Osteogenic graft material treated groups and the non-OP-1® Osteogenic graft material groups was very obvious. In addition, the direction of newly formed bone was irregular, which made uniform orientation of tissue sectioning very difficult, and that orientation is a prerequisite for quantitative analysis. The osteo-induction of BMP has been demonstrated previously, although the activity varied with different carriers. In this study, no carrier for OP-1® Osteogenic graft material delivery was used for the group not implanted with Ti disks. That approach not only confirms the reproductibility of the osteoinductive activity of OP-1® Osteogenic graft material, but also provides a simple and reliable model for a bone induction assay without the interference from a carrier.

Histological findings at 3 weeks still showed the bioactivity of OP-1® Osteogenic graft material, which usually takes place at the early stage of bone induction. Evidence of chemotaxis, angiogenesis and chondrogenesis was observed. This BMP-induced bone formation with a cartilaginous process has been observed in several studies in which different carriers influenced only the amount and the time-dependent occurrence of cartilage. The hypertrophic chondrocytes which were found in some of the tissue sections are probably the residues of early cellular response to OP-1® Osteogenic graft material and not the result of the remaining OP-1® Osteogenic graft material, because the mean residence time of BMP in tissue is less than 4 days.

Most of the surface of the disks with OP-1® Osteogenic graft material was surrounded by trabecular bone, which was not in close contact with the implants but was separated by bone marrow tissue. This is most likely the consequence of movement of the disk in the muscle pouch, where the disk was not tightly fit. Based on the high rate of bone induction in this ectopic location, it is reasonable to expect that the same composite of a PA-coated implant and OP-1® Osteogenic graft material will induce bone formation more easily in an orthotopic osseous environment, where most osteoprogenitor cells are available as the target cells for OP-1® Osteogenic graft material. Marrow stromal cells and pericytes around capillaries and venules have been shown to be such target cells. They can be induced to differentiate towards osteoblasts. More animal studies of orthotopic osseous formations are needed in order to establish the role of OP-1® Osteogenic graft material when carried by the PA enhanced coating of an implant.

Thus, the results of Example III indicate the osteoinductive activity of OP-1® Osteogenic graft material is maintained after loading on PA-coated Ti disks. This method of delivering OP-1® Osteogenic graft material may provide an approach for applying OP-1® Osteogenic graft material to the surface of cementless endoprosthetic components.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of applying coating having a therapeutic agent to an implant comprising:
coating the implant with a layer of crystalline hydroxyapatite by precipitating the hydroxyapatite from solution;
drying the coated implant;
sterilizing the coated implant;
packaging the coated implant;
removing the implant from the package and then coating the implant with OP-1 protein in a solution; and
thereafter implanting the implant coated with the therapeutic agent.

2. The method as set forth in claim 1 further including implanting the implant in its wetted state.

3. The method as set forth in claim 1 wherein the OP-1 protein is dissolved in a lactose solution.

4. The method as set forth in claim 1 wherein the OP-1 protein is suspended in a lactose solution.

5. The method as set forth in claim 1 wherein the implant is immersed in the OP-1 protein solution.

6. The method as set forth in claim 1 wherein the OP-1 protein solution is added to the dried coating drop wise.

7. The method as set forth in claim 1 wherein the hydroxyapatite coating comprises at least two layers.

8. A method for providing a therapeutic agent to an implant comprising:
providing an implant having a dry coating of crystalline calcium phosphate minerals;
packaging the implant;
sterilizing the implant;
removing the implant from the package;
coating the crystalline calcium phosphate implant after removing it from the package with an aqueous solution of a therapeutic agent comprising OP-1 protein in a solution by adding the solution to the dried coating drop wise; and
immediately implanting the implant coated with the solution of the therapeutic agent.

9. The method as set forth in claim 8 wherein the solution is a lactose solution.

10. The method as set forth in claim 9 wherein the therapeutic agent is dissolved in the lactose solution.

11. The method as set forth in claim 9 wherein the therapeutic agent is suspended in the lactose solution.

12. The method as set forth in claim 8 wherein the implant is immersed in the OP-1 protein solution.

13. The method as set forth in claim 8 wherein the coating of crystalline calcium phosphate has more than one layer.

14. A method of applying a coating having a therapeutic agent to an implant comprising:
coating the implant with a layer of hydroxyapatite by precipitating the hydroxyapatite from solution;
drying the coated implant;
packaging the dried coated implant;
sterilizing the packaged coated implant;
mixing a desired therapeutic agent with water;
removing the dried implant from the package;
coating the dried hydroxyapatite coated implant with a solution of OP-1 protein; and
immediately implanting the implant after coating it with the OP-1 protein solution.

15. The method as set forth in claim 14 further including implanting the implant in its wetted state.

16. The method as set forth in claim 14 wherein the therapeutic agent is dissolved in a lactose solution.

17. The method as set forth in claim 16 wherein the therapeutic agent is suspended in the lactose solution.

18. The method as set forth in claim 16 wherein the implant is immersed in the lactose solution.

19. The method as set forth in claim 14 wherein the OP-1 protein solution is added to the dried coating drop wise.

20. The method as set forth in claim 14 wherein the hydroxyapatite coating is comprises at least two layers.
